(19) United States
(12) Patent Application Publication
(10) Pub. No.: US 2018/0161376 A1
(43) Pub. Date: Jun. 14, 2018

(54) COMPOSITIONS AND METHODS FOR NEURONAL DIFFERENTIATION OF CELLS

(71) Applicant: Northern Sydney Local Health District, St. Leonards (AU)

(72) Inventors: Carolyn Mary SUE, Lane Cove (AU); Steven GAO, St. Leonards (AU); Nicholas BLAIR, Balmain (AU)

(21) Appl. No.: 15/533,877

(22) PCT Filed: Dec. 8, 2015
(86) PCT No.: PCT/AU2015/000739

§ 371 Date: Jun. 7, 2017

(30) Foreign Application Priority Data
Dec. 8, 2014 (AU) 2014904953

(51) Int. Cl. A61K 35/30; G01N 33/50; C12N 5/0793; C12N 5/0797

(52) U.S. Cl. CPC A61K 35/30; G01N 33/5058; C07K 14/70571; C12N 5/0623; G01N 2800/28; C12N 5/0619

(57) ABSTRACT

This invention relates to compositions and methods for enhanced differentiation of cells towards a neuronal phenotype. Neuronal cells produced using the compositions and methods may be used in the study of neurological diseases and disorders, for drug screening and for therapeutic purposes. The invention provides a method for producing a neuron comprising inducing neuronal differentiation of a cell, wherein neuronal differentiation in said cell is induced by inhibition of Small Mothers Against Decapentaplegic (SMAD) signaling and nitric oxide synthase (NOS) in said cell.

### FIELD

This invention relates to compositions and methods for enhanced differentiation of cells towards a neuronal phenotype. Neuronal cells produced using the compositions and methods may be used in the study of neurological diseases and disorders, for drug screening and for therapeutic purposes.

### BACKGROUND

Investigation of human disease is best performed using an affected tissue. However, it is impossible to obtain or culture a patient's neurons for the study of a neurodegenerative disease. Thus, cell and animal models are essential to neurological research. Animal models do not necessarily reflect human disease processes accurately and cell models of neurological disease have historically been either non-human or non-neuronal (e.g. PC12 and COS7 (Valente, Abou-Sleiman et al. 2004)) and/or are derived from malignant cell lines such that they can be grown in vitro (e.g. human derived-neuroblastoma-derived cell lines (SH-SY5Y) (Imai, Soda et al. 2001, Valente, Abou-Sleiman et al. 2004)). Thus, it is imperative to develop faithful, human-derived, biologically relevant cell models to investigate neurologic diseases.

Alternative sources of neurons include those differentiated from stem cells, including those present in the human olfactory mucosa. Stern cells can be isolated from the olfactory mucosa from adult humans (Murrell, Feron et al. 2005, Wetzig, Mackay-Sim et al. 2011). Populations of these cells have been shown to be multipotent in an in vitro and in vivo environment (Murrell, Feron et al. 2005). Olfactory stem cells have been used as neuronal disease models for Parkinson's disease (Murrell, Wetzig et al. 2008), ataxia-telangiectasia (Stewart, Kozlov et al. 2013) and hereditary spastic paraplegia (Abrahamsen, Fan et al. 2013). However, despite the advances of disease modeling using olfactory neurospheres, there has been limited success in differentiating these cells into mature neuronal cells. The neural progenitor cells that constitute olfactory neurospheres may not recapitulate the function of a mature neuron and thus it is important to develop methods to effectively differentiate olfactory neural stem cells into neurons in order to establish a more faithful neuronal model.

Additionally, obtaining neuronal cells suitable for transplantation and screening of candidate therapeutic agents will have great use in the treatment of neurodegenerative diseases, such as amyotrophic lateral sclerosis, Alzheimer's disease, Huntington's disease and Parkinson's disease. Current methods for the in vitro differentiation of neurons are also time intensive. Accordingly, culture conditions that achieve rapid differentiation of stern cells to functional neuronal cells that mimic in vivo differentiation would be desirable.

### SUMMARY OF INVENTION

The present invention relates generally to the field of cell biology of stem cells, more specifically the directed differentiation of stem cells and progenitor cells using novel culture conditions.

According to one aspect, the present invention provides a method for producing a neuron comprising inducing neuronal differentiation of a cell, wherein neuronal differentiation in said cell is induced by inhibition of Small Mothers Against Decapentaplegic (SMAD) signaling and nitric oxide synthase (NOS) in said cell.

In one embodiment, inhibition of SMAD signaling occurs by contacting a cell with at least two SMAD inhibitors and inhibition of NOS occurs by contacting the cell with at least one NOS inhibitor.

In one embodiment, the at least two SMAD inhibitors are selected from any two or more of (SB431541), 4-[4-(1,3-benzodioxol -5-yl)-5-(2-pyridinyl)-1H-imidazol-2-yl]benzamide (SB431542), 4-(6-(4-(piperazin-1-yl)phenyl)pyrazolo[1,5-a]pyrimidin-3-yl)quinolone hydrochloride (LDN193189), 2-(4-(benzo[d][1,3]dioxol-5-yl)-2-tert-butyl-1H-imidazol-5-yl)-6-methylpyridine (SB505124), 4-[2-(6-Methyl-pyridin-2-yl)-5,6-dihydro-4H-pyrrolo[1,2-b]pyrazol-3-yl]-quinoline-6-carboxylic acid amide (LY2157299), 4-[6-(4-Isopropoxyphenyl) pyrazolo[1,5-a]pyrimidin-3-yl]quinoline, 4-[6-[4-(1-Methylethoxy)phenyl]pyrazolo[1,5-a]pyrimidin-3-yl]-quinoline (DMH1), (2E)-1-(6,7-Dimethoxy-3,4-dihydro-1H-isoquinolin-2-yl)-3-(1-methyl-2-phenyl-1H-pyrrolo[2,3-b]pyridin-3-yl)-propenone hydrochloride (SIS3) and Noggin.

In another embodiment, the at least one NOS inhibitor is selected from any one of Diphenyleneiodonium Chloride, Dexamethasone, 1-Pyrrolidinecarbodithioic Acid, 7-Nitroindazole, 1400W, 1-Amino-2-hydroxyguanidine, p-Toluenesulfonate, S-Methylisothiourea, S,S′-1,3-Phenylene-bis(1,2-ethanediyl)-bis-isothiourea.2HBr (1,3-PBITU), N6-(1-iminoethyl)-L-lysine (L-NIL), 1-(2-Trifluoromethylphenyl) Imidazole (TRIM), N-(1,4-dihydro-1,4-dioxo-2-naphthalenyl)-benzamide (PPM-18), The Nitric Oxide Synthase Neuronal Inhibitor I, Chlorpromazine, Spermidine, N^{G}-Nitro-L-arginine, Aminoguanidine, S-Methyl-L-thiocitrulline, S-Methylisothiourea, Zinc (II) Protoporphyrin IX, Mercaptoethylguanidine (MEG), Bromocriptine Mesylate, Melatonin, L-Thiocitrulline, N^{G},N^{G}-Dimethyl-L-arginine, N^{G}-Propyl-L-arginine, α-phenyl-α-propyl-2-(diethylamino)ethyl ester-benzeneacetic (SKF-525A, Proadifen), Haloperidol, N^{G}-Monomethyl-D-arginine, 2-Ethyl-2-thiopseudourea, L-N⁵-(1-Iminoethyl/ornithine, Caveolin-1 Scaffolding Domain Peptide and p-Nitroblue Tetrazolium Chloride.

In another embodiment, the at least two SMAD inhibitors are SB431542 and LDN-193189 and the nitric oxide synthase inhibitor is TRIM.

In one embodiment, the cell is isolated from a human. In another embodiment the cell is selected from the group consisting of a stem cell, progenitor cell, a dedifferentiated cell, neural stem cell, neural progenitor cell, or primary olfactory cell. In another embodiment, the cell is a primary olfactory cell from an olfactory neurosphere. In another embodiment, the cell has or is modified to have i) inhibited expression of a gene product of interest, ii) expression of a gene product of interest with impaired function, iii) increased expression or overexpression of a gene product of interest or iv) expression of a gene product of interest with enhanced function.

In another aspect, the present invention provides a method for producing a neuron by inducing neuronal differentiation in one or more cells of an olfactory neurosphere comprising the steps of: (i) culturing one or more primary olfactory cells from a subject under conditions to form an olfactory neurosphere; (ii) isolating said neurosphere; and (iii) inducing neuronal differentiation in one or more cells within the neurosphere by inhibiting SMAD signaling and NOS.

According to another aspect, the present invention provides a method for producing a neuron comprising the steps of: obtaining one or more primary olfactory cells from a subject; culturing said one or more cells in culture conditions to form an olfactory neurosphere; isolating said neurosphere; and inducing neuronal differentiation in one or more cells of said neurosphere by culturing said one or more cells under conditions which inhibit SMAD signaling and NOS; wherein neuronal differentiation is achieved after about 3 to 4 days following culture under conditions which inhibit SMAD signaling and NOS.

According to another embodiment, the present invention provides a method for inducing neuronal differentiation in a cell comprising the steps of: (i) culturing one or more primary olfactory cells from a subject in culture conditions to form an olfactory neurosphere; (ii) isolating said neurosphere; and (iii) culturing said neurosphere in a medium comprising at least two SMAD inhibitors selected from SB431541, SB431542, LDN193189, SB505124, LY2157299, DMH1, SIS3 and Noggin and at least one NOS inhibitor selected from any one of Diphenyleneiodonium Chloride, Dexamethasone, 1-Pyrrolidinecarbodithioic Acid, 7-Nitroindazole, 1400W, 1-Amino-2-hydroxyguanidine, p-Toluenesulfonate, S-Methylisothiourea, 1,3-PBITU, L-NIL, TRIM, PPM-18, The Nitric Oxide Synthase, Neuronal Inhibitor I, Chlorpromazine, Spermidine, N^{G}-Nitro-L-arginine, Aminoguanidine, S-Methyl-L-thiocitrulline, S-Methylisothiourea, Zinc (II) Protoporphyrin IX, MEG, Bromocriptine Mesylate, 1,3-PBITU, Melatonin, L-Thiocitrulline, N^{G},N^{G}-Dimethyl-L-arginine, N^{G}-Propyl-L-arginine, SKF-525A, Haloperidol, N^{G}-Monomethyl-D-arginine, 2-Ethyl-2-thiopseudourea, L-N⁵-(1-Iminoethyl/ornithine, Caveolin-1 Scaffolding Domain Peptide and p-Nitroblue Tetrazolium Chloride.

According to another embodiment, the present invention provides a method for inducing neuronal differentiation in a cell comprising the steps of: (i) culturing one or more primary olfactory cells from a subject in culture conditions to form an olfactory neurosphere; (ii) isolating said neurosphere; and (iii) culturing said neurosphere in a medium comprising at least two SMAD inhibitors SB431542 and LDN-193189 and the nitric oxide synthase inhibitor TRIM; wherein neuronal differentiation of said cell occurs after about 3 to 4 days of step (iii).

In one embodiment, the one or more primary olfactory cells is obtained from an olfactory biopsy.

According to another aspect, the present invention provides a neuron produced according to a method of the invention described herein. According to another embodiment, a neuron produced according to the present invention has, or may be modified to have i) inhibited expression of a gene product of interest, ii) expression of a gene product of interest with impaired function, iii) increased expression or overexpression of a gene product of interest or iv) expression of a gene product of interest with enhanced function.

According to another aspect, the present invention provides a use of a neuron produced according to the methods of the present invention for the prevention or treatment of a neurodegenerative disorder or a disorder of the peripheral nervous system in a subject.

According to another aspect, the present invention provides a use of a neuron produced according to the methods of the present invention for the regeneration or repair of the CNS or peripheral nervous system.

According to another aspect, the present invention provides a use of a neuron produced according to the methods of the present invention in a method of screening or identification of a neurotoxic agent or an agent useful for the prevention or treatment of a neurodegenerative disorder or a disorder of the peripheral nervous system in a subject.

According to another aspect, the present invention provides a method of identification of an agent useful for the prevention or treatment of a neurodegenerative disorder in a subject or regeneration or repair of the CNS or peripheral nervous system comprising: (a) contacting a neuron produced according to the methods of the invention with an agent; and (b) detecting an increase or decrease in a parameter relative to a control cell not contacted with the agent; wherein said increase or decrease in the parameter is indicative of a neuroprotective or therapeutic effect; and wherein an agent that effects said increase or decrease is identified as being useful for the treatment of a neurodegenerative disorder or regeneration or repair of the CNS or peripheral nervous system.

According to another aspect, the present invention provides a method of identification of an agent having a neurotoxic effect, comprising: (a) contacting a neuron produced according to the methods of the invention with an agent; and (b) detecting an increase or decrease in a parameter relative to a control cell not contacted with the agent; wherein said increase or decrease in the parameter is indicative of a neurotoxic effect; and wherein an agent that effects said increase or decrease is identified as having a neurotoxic effect.

According to the another embodiment, a neuron produced according to the present invention may be modified to have inhibited expression of a gene product of interest, to express a gene product of interest with impaired function, or modified to have corrected expression of a gene product of interest, to overexpress a gene product of interest or express a gene product of interest with enhanced function and used in a method of screening or identification of a neurotoxic agent or an agent useful for the prevention or treatment of a neurodegenerative disorder or a disorder of the peripheral nervous system in a subject.

According to another embodiment, the invention provides a cell culture medium for the neuronal differentiation of a cell, comprising at least two SMAD inhibitors and at least one NOS inhibitor.

According to another embodiment, the invention provides a kit for the neuronal differentiation of a cell, comprising at least two SMAD inhibitors and at least one NOS inhibitor.

In another embodiment, the medium or kit according to the previous embodiments comprises at least two SMAD inhibitors selected from SB431541, SB431542, LDN193189, SB505124, LY2157299, DMH1, SIS3 and Noggin and at least one NOS inhibitor selected from SB431541, SB431542, LDN193189, SB505124, LY2157299, DMH1, SIS3 and Noggin and at least one NOS inhibitor selected from any one of Diphenyleneiodonium Chloride, Dexamethasone, 1-Pyrrolidinecarbodithioic Acid, 7-Nitroindazole, 1400W, 1-Amino-2-hydroxyguanidine, p-Toluenesulfonate, S-Methylisothiourea, 1,3-PBITU, L-NIL, TRIM, PPM-18, The Nitric Oxide Synthase, Neuronal Inhibitor I, Chlorpromazine, Spermidine, N^{G}-Nitro-L-arginine, Aminoguanidine, S-Methyl-L-thiocitrulline, S-Methylisothiourea, Zinc (II) Protoporphyrin IX, MEG, Bromocriptine Mesylate, Melatonin, L-Thiocitrulline, N^{G},N^{G}-Dimethyl-L-arginine, N^{G}-Propyl-L-arginine, SKF-525A, Haloperidol, N^{G}-Monomethyl-D-arginine, 2-Ethyl-2-thiopseudourea, L-N⁵-(1-Iminoethyl)ornithine, Caveolin-1 Scaffolding Domain Peptide andp-Nitroblue Tetrazolium Chloride.

In another embodiment, the medium or kit comprises the SMAD inhibitors SB431542 and LDN-193189 and the nitric oxide synthase inhibitor TRIM.

In another embodiment, the kit further comprises a cell selected from the group consisting of a stem cell, progenitor cell, a dedifferentiated cell, neural stem cell, neural progenitor cell, or primary olfactory cell. In another embodiment, the cell is a primary olfactory cell is from an olfactory neurosphere.

According to another embodiment, a kit of the present invention further comprises an agent for detecting expression of one or more markers of neuronal differentiation. In another embodiment, the invention further comprises a cell culture medium.

### BRIEF DESCRIPTION OF DRAWINGS

FIG. 1A shows the effects of different combinations of reagents on the differentiation of olfactory primary cultures; FIG. 1B shows the characterization of olfactory primary cultures by immunostaining.

FIG. 2 shows induction of olfactory neurospheres (ONS) from olfactory primary cultures and their characterization.

FIG. 3 shows the effects of combined treatment of SB431542, LDN-193189 and TRIM on ONS.

FIG. 4 shows characterization of ONS derived neurons.

### DESCRIPTION OF EMBODIMENTS

Definitions

As used herein, the term “cell” refers to a single cell as well as to a population of (i.e., more than one) cells. The population may be a pure population comprising one cell type, such as a population of neuronal cells or a population of undifferentiated embryonic cells. Alternatively, the population may comprise more than one cell type, for example a mixed cell population. It is not meant to limit the number of cells in a population, for example, a mixed population of cells may comprise at least one differentiated cell. In one embodiment a mixed population may comprise at least one differentiated. In the present inventions, there is no limit on the number of cell types that a cell population may comprise.

As used herein, the term “differentiation” as used with respect to cells in a differentiating cell system refers to the process by which cells differentiate from one cell type (e.g., a multipotent, totipotent or pluripotent differentiable cell) to another cell type such as a target differentiated cell). Accordingly, the term “cell differentiation” as used herein, refers to a specialization process or a pathway by which a less specialized cell (e.g. stem cell) develops or matures to possess a more distinct form and function (i.e. more specialized).

As used herein, the term “dedifferentiation” or “dedifferentiated” as used with respect to cells, refers to a process wherein a more specialized cell having a more distinct form and function, and/or limited self-renewal and/or proliferative capacity becomes less specialized and acquires a greater self-renewal and/or proliferative capacity or differentiation capacity (e.g. multipotent, pluripotent etc.). An induced Pluripotent Stem Cell (iPSC) is an example of a dedifferentiated cell. Accordingly, dedifferentiation can refer to a process of cellular reprogramming.

As used herein, the term “inducing neuronal differentiation” in reference to a cell refers to changing the default cell type (genotype and/or phenotype) to a non-default cell type (genotype and/or phenotype). Thus “inducing neuronal differentiation in a cell” includes inducing a cell to have neuronal characteristics, or inducing a cell to divide into progeny cells with neuronal characteristics, that are different from the original identity of the cell, such as genotype (i.e. change in gene expression as determined by genetic analysis such as a PCR or microarray) and/or phenotype (i.e. change in morphology, function and/or expression of a protein, such as β-III tubulin or a plurality of proteins, including a combination of two or more of β-III tubulin, Microtubule Associated Protein 2 (MAP2), synapsin, neurofilament-L, Nestin and N-Cam).

As used herein, the term “neuron” refers to a differentiated, lineage committed cell of the neural lineage that exhibits the functional and/or phenotypical characteristics of a mature post-mitotic neuron, or a differentiated, lineage committed cell of the neural lineage that requires further maturation, either in vivo or in vitro, in order to exhibit the functional and/or phenotypical characteristics of a mature post-mitotic neuron. Neurons can express one or more of the following markers: β-III tubulin, Microtubule Associated Protein 2 (MAP2), Synapsin, Neurofilament-L, Nestin and N-Cam.

As used herein, the term “inhibit”, “inhibiting” and “inhibition” refers to a reduction, decrease, inactivation, down-regulation, elimination or suppression of an activity or quantity. Accordingly, as used herein, the term “inhibitor” refers to an agent that interferes with (i.e. reduces, decreases, inactivates, down-regulates, eliminates or suppresses) the gene or protein expression of a molecule and/or the activity and/or function of a molecule. For example, in reference to inhibiting a signaling molecule or a signaling molecule's pathway, such as an inhibitor of SMAD signaling, an inhibitor refers to refers to an agent that interferes with the gene or protein expression of an entity involved in the SMAD signaling pathway and/or the activity and/or function of a signaling molecule or the signaling function of the molecule or pathway. Similarly, in reference to an inhibitor of NOS, an inhibitor refers to an agent which interferes with the expression or activity or function of NOS.

As used herein, the term “contacting” cells with a compound as defined by the present inventions refers to placing the compound in a location that will allow it to touch the cell in order to produce “contacted” cells. The contacting may be accomplished using any suitable method. For example, in one embodiment, contacting is by adding the compound to a container (e.g. tube, vial or culture flask or culture dish etc.) of cells. Contacting may also be accomplished by adding the compound to a culture of the cells.

As used herein, the term “stem cell” refers to a cell that is totipotent or pluripotent or multipotent and are capable of differentiating into one or more different cell types, such as embryonic stems cells, stem cells isolated from organs, for example, olfactory neural stem cells. As used herein, the term “adult stem cell” refers to a stem cell derived from an organism after birth.

As used herein, the term “neural stem cell” or “NSC” or “neural precursor cell” or “neural progenitor cell” refers to a cell that is capable of becoming neurons, astrocytes, oligodendrocytes, and glial cells in vivo, and neuronal cell progeny and glial progeny in culture.

As used herein, the term “neural cell line” refers to a cell line displaying characteristics normally associated with a neural cell. Examples of such characteristics include, but are not limited to, expression of FOXA2, SHH, Netrin-1, F-Spondin, and the like.

As used herein, the term “pluripotent” refers to a cell line capable of differentiating into any (or multiple) differentiated cell type(s).

As used herein, the term “multipotent” refers to a cell line capable of differentiating into at least two differentiated cell types.

As used herein, the term “primary cell” is a cell that is directly obtained from a tissue (e.g. blood) or organ of an animal in the absence of culture. Typically, though not necessarily, a primary cell is capable of undergoing ten or fewer passages in vitro before senescence and/or cessation of proliferation.

As used herein, the term “cell line,” refers to cells that are cultured in vitro, including primary cell lines, finite cell lines, continuous cell lines, and transformed cell lines, but does not require, that the cells be capable of an infinite number of passages in culture. Cell lines may be generated spontaneously or by transformation.

As used herein, the term “cell culture” refers to any in vitro culture of cells. The term “culturing” refers to the process of growing and/or maintaining and/or manipulating a cell. Included within this term are continuous cell lines (e.g., with an immortal phenotype), primary cell cultures, finite cell lines (e.g., non-transformed cells), and any other cell population maintained in vitro, including oocytes and embryos. As used herein, the terms “primary cell culture,” and “primary culture,” refer to cell cultures that have been directly obtained from cells in vivo, such as from a tissue specimen or biopsy from an animal or human. These cultures may be derived from adults as well as fetal tissue.

As used herein, the terms “culture medium,” and “cell culture medium,” refer to media that are suitable to support the growth of cells in vitro (i.e., cell cultures, cell lines, etc.). It is not intended that the term be limited to any particular culture medium. For example, it is intended that the definition encompass maintenance media as well as other media for the differentiation or specialization of cells. Indeed, it is intended that the term encompass any culture medium suitable for the growth of the cell cultures and cells of interest.

As used herein, the term “kit” refers to any delivery system for delivering materials. In the context of cell differentiation, a kit may refer to a combination of materials for contacting stem cells, such delivery systems include systems that allow for the storage, transport, or delivery of reaction reagents (e.g., compounds, proteins, detection agents (such as probes or antibodies), etc. in the appropriate containers (such as tubes, etc.) and/or supporting materials (e.g., buffers, written instructions for performing cell differentiation, etc.) from one location to another. For example, kits include one or more enclosures (e.g., boxes, or bags, and the like) containing the relevant reaction reagents (such as SMAD inhibitors (e.g. SB431542 (or a SB431542 replacement) and LDN193189 (or an LDN193189 replacement, etc.) and NOS inhibitors (e.g. TRIM (or a TRIM replacement)) and/or supporting materials.

As used herein, the term “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reaction that occur within a natural environment.

As used herein, the term “marker” or “cell marker” refers to gene or protein that identifies a particular cell or cell type. A marker for a cell may not be limited to one marker; markers may refer to a “pattern” of markers such that a designated group of markers may identify a cell or cell type from another cell or cell type. For example, neurons of the present inventions express one or more markers that distinguish a neuron cell, e.g. β-III tubulin, Nestin and N-Cam.

The term “derived from” or “established from” or “differentiated from” when made in reference to any cell disclosed herein refers to a cell that was obtained from (e.g., isolated, purified, etc.) a parent cell in a cell line, tissue (such olfactory mucosa), or fluids using any manipulation, including single cell isolation, in vivo culture, treatment and/or mutagenesis using for example proteins, chemicals, radiation, infection with virus, transfection with DNA sequences, such as with a morphagen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

As used herein, the terms “neurodegenerative disorder” and “neurodegenerative disease” are used interchangeably in this document and mean diseases of the nervous system (e.g., the central nervous system or peripheral nervous system) characterized by abnormal cell death. Examples of neurodegenerative conditions include Alzheimer disease, Down's syndrome, frontotemporal dementia, progressive supranuclear palsy, Pick's disease, Niemann-Pick disease, Parkinson's disease, Huntington's disease, dentatorubropallidoluysian atrophy, Kennedy's disease (also referred to as spinobulbar muscular atrophy), and spinocerebellar ataxia (e.g., type 1 , type 2, type 3 (also referred to as Machado-Joseph disease), type 6, type 7, and type 17)), fragile X (Rett's) syndrome, fragile XE mental retardation, Friedreich's ataxia, myotonic dystrophy, spinocerebellar ataxia type 8, and spinocerebellar ataxia type 12, Alexander disease, Alper's disease, amyotrophic lateral sclerosis (or motor neuron disease), Hereditary spastic paraplegia, mitochondrial disease, ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemic stroke, Krabbe disease, Lewy body dementia, multiple sclerosis, multiple system atrophy, Pelizaeus-Merzbacher disease, Pick's disease, primary lateral sclerosis, Refsum's disease, Sandhoff disease, Schilder's disease, spinal cord injury, spinal muscular atrophy, Steele-Richardson-Olszewski disease, and Tabes dorsalis.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components, or group thereof.

A reference herein to a patent document or other matter which is given as prior art is not to be taken as an admission that that document or matter was known or that the information it contains was part of the common general knowledge as at the priority date of any of the claims.

Differentiation of Neural Stem and Progenitor Cells

Human stem cells offer great promise for cell-replacement therapies and cell screening for therapeutics. Recent advances in somatic cell reprogramming to induced pluripotent stem cells (iPSCs) has opened the door to generating patient-specific cells for regenerative medicine and disease modeling. However to realize the full potential of these approaches for disorders of the central and peripheral nervous systems, improved differentiation protocols are required that eliminate the use of undefined factors and increase the speed of differentiation, which can require as long as 40+ days, as well as the and efficiency of differentiation. Accordingly, understanding and manipulating the signaling pathways involved in neural differentiation of stem cells is critical.

Neural stem or progenitor cells and neural subtypes as derived from the same have been the focus of numerous scientific publications and patent applications. However, the generation of neural stem/progenitor cells from embryoid body formation, from human embryonic stem cells and from iPSCs and the neuronal differentiation of these stem/progenitor cells is labor and resource intensive and frequently involves the use of feeder cells and other undefined factors.

Various agents for neuronal differentiation include growth factors of various kinds, such as epidermal growth factor (EGF), transforming growth factor β (TGF-β), any type of fibroblast growth factor (exemplified by FGF-4, FGF-8, and basic fibroblast growth factor (bFGF)), platelet-derived growth factor (PDGF), insulin-like growth factor (IGF-1 and others), high concentrations of insulin, sonic hedgehog, members of the neurotrophin family (such as nerve growth factor (NGF), neurotrophin 3 (NT-3), brain-derived neurotrophic factor (BDNF)), bone morphogenic proteins (especially BMP-2 & BMP-4), retinoic acid (RA) and ligands to receptors that complex with gp130 (such as LIF, CNTF, and IL-6). Also known are alternative ligands and antibodies that bind to the respective cell-surface receptors for the aforementioned factors. Typically, a plurality of differentiation agents is used, which may comprise 2, 3, 4, or more of the agents listed above.

The inventors have demonstrated that rapid and efficient neuronal differentiation can be achieved when SMAD signaling is inhibited and NOS is inhibited in neural stem or progenitor cells.

The inventors have demonstrated that rapid and efficient neuronal differentiation can be achieved when neural stem or progenitor cells are contacted with a combination of two SMAD inhibitors and at least one NOS inhibitor.

According to one embodiment, the present invention provides a method of inducing the neuronal differentiation of a neural stem or progenitor cell by culturing the cell in a cell culture medium which comprises SB431542 and LDN193189 and TRIM.

The amount of each of the SMAD inhibitors and the NOS inhibitor required to be supplied to a cell for the induction of neuronal differentiation may be readily determined by the person skilled in the art. For example, the level of inhibition of SMAD signaling and/or expression and the level of NOS activity and/or expression may be determined by routine assays. The concentrations of SMAD and NOS inhibitors to be used in the methods and compositions (including but not limited to cell culture media and kits) may be readily ascertained having regard to neuronal differentiation and optionally cell viability, both of which may be readily assessed using assays known to the skilled addressee together with the assays described herein, and adjusted accordingly.

In one embodiment, SB431542 is added to the culture medium in a concentration ranging from 5 to 100 μM, preferably ranging from 5 to 20 μM, even more preferably at about 10 μM. Typically, LDN193189 is added to the culture medium in a concentration ranging from 5 to 500 nM, preferably ranging from 75 to 150 nM, even more preferably at about 100 nM. Typically, TRIM is added to the culture medium in a concentration ranging from 10 to 1000 μM, preferably ranging from 50 to 200 μM, even more preferably at about 100 μM.

In one embodiment, the present invention provides a method of wherein induction of neuronal differentiation of a neural stem or progenitor cell is associated with inhibition of expression of a SMAD gene in a cell. In one embodiment the expression of SMAD4 is inhibited. In one embodiment, the level of SMAD4 gene expression is suppressed so as to provide at least 80% inhibition of SMAD4 expression compared to a cell not contacted with at least two SMAD inhibitors. Preferably, the level of inhibition is at least 81%, 82%, 83%, 84%, 85%, 86%, 87%, 88%, 89%, 90%, 91% or at least 92%.

The inventors have further demonstrated that by using olfactory neurospheres (ONS) as a starting material, rapid and efficient neuronal differentiation of cells from ONS can be obtained. Whereas current methods employed in the art are inefficient and require up to 10 days culture to obtain neuronal phenotype by culturing expanded spheres in neurobasal media supplemented with growth factors not inhibitors. Using the methods of the present invention, neurons exhibiting typical neuronal morphology and expression of neuronal markers such as β-III-tubulin, Synapsin and Neurofilament-L) can be obtained in a much shorter period of time.

Accordingly, in one embodiment, one or more neurons may be produced within about 7, about 6, about 5, about 4, or about 3 days of one or more neural stem or progenitor cell(s) being cultured under conditions wherein SMAD signaling is inhibited and NOS is inhibited. In certain embodiments, one or more neurons are produced from one or more neural stern or progenitor cell(s) within about 7, about 6, about 5, about 4, or about 3 days of the cell(s) being cultured in the presence of SB431542, LDN193189 and TRIM.

In one embodiment, the cell is from an olfactory neurosphere. In certain embodiments, neurons are produced from olfactory neurospheres (ONS) within about 7, about 6, about 5, about 4, or about 3 days of the ONS being cultured in the presence of SB431542, LDN193189 and TRIM.

Typically, almost all cells from a young small olfactory neurosphere exhibited the characteristics of neurons after being subjected to the differentiation described herein. The efficiency of neuronal differentiation may be determined by counting the total number of cells of an olfactory neurosphere and the number thereof which adopt a neuronal phenotype. In one embodiment, the efficiency of differentiation is at least 40%, 50%, 60%, 70%, 80% and preferably at least 83%.

The neuronal differentiation can take place in a culture environment comprising a suitable substrate, and a nutrient medium to which the differentiation agents are added. Suitable substrates include solid surfaces coated with a positive charge, such as a basic amino acid, exemplified by poly-L-lysine and polyornithine. Substrates can be coated with extracellular matrix components, exemplified by fibronectin. Other permissive extracellular matrices include Matrigel® (extracellular matrix from Engelbreth-Holm-Swarm tumor cells) and laminin. Also suitable are combination substrates, such as poly-L-lysine (PLL) or poly-D-Lysine (PDL) combined with fibronectin or laminin, or both.

According to one embodiment, neuronal differentiation of the cell takes place on a surface coated with poly-D-lysine and laminin.

The neural stem or progenitor cells or primary olfactory cells used in the methods of the invention may be isolated from healthy subject or a subject characterized as having a neurodegenerative disorder or a disorder of the peripheral nervous system.

Cell Culture Medium

The present invention also provides a cell culture medium for the inducing neuronal differentiation. Typically a cell culture medium includes a source of carbon as energy substrate, such as glucose, galactose or sodium pyruvate; essential amino acids; vitamins, such as biotin, folic acid, B12; at least a purine and a pyrimidine as nucleic acid precursors; and inorganic salts.

The culture medium may also optionally include other supplements selected from the group including but not limited to antibiotics, antimycotics, growth factors, inhibitors, epigenetic modifiers, mRNA and miRNA.

The culture medium of the invention may also comprise various supplements such as the B27 supplement contains, amongst other constituents, SOD, catalase and other anti-oxidants (GSH), and unique fatty acids, such as linoleic acid, linolenic acid, and lipoic acids.

The culture medium may also contain pH buffers in order to maintain the pH of the medium at a value suitable for cell growth. The culture medium of the invention may be based on a commercially available medium such as DMEM/F12 or a mixture of DMEM/F12 and Neurobasal medium in a 1:1 ratio.

According to one embodiment, the invention provides a cell culture medium for the neuronal differentiation of a cell, comprising at least two SMAD inhibitors and at least one NOS inhibitor.

In one embodiment, the at least two SMAD inhibitors selected from SB431541, SB431542, LDN193189, SB505124, LY2157299, DMH1, SIS3 and Noggin and at least one NOS inhibitor selected from SB431541, SB431542, LDN193189, SB505124, LY2157299, DMH1, SIS3 and Noggin and at least one NOS inhibitor selected from any one of Diphenyleneiodonium Chloride, Dexamethasone, 1-Pyrrolidinecarbodithioic Acid, 7-Nitroindazole, 1400W, 1-Amino-2-hydroxyguanidine, p-Toluenesulfonate, S-Methylisothiourea, 1,3-PBITU, L-NIL, TRIM, PPM-18, The Nitric Oxide Synthase, Neuronal Inhibitor I, Chlorpromazine, Spermidine, N^{G}-Nitro-L-arginine, Aminoguanidine, S-Methyl-L-thiocitrulline, S-Methylisothiourea, Zinc (II) Protoporphyrin IX, MEG, Bromocriptine Mesylate, Melatonin, L-Thiocitrulline, N^{G},N^{G}-Dimethyl-L-arginine, N^{G}-Propyl-L-arginine, SKF-525A, Haloperidol, N^{G}-Monomethyl-D-arginine, 2-Ethyl-2-thiopseudourea, L-N⁵-(1-Iminoethyl)ornithine, Caveolin-1 Scaffolding Domain Peptide and p-Nitroblue Tetrazolium Chloride.

In a preferred embodiment, the culture medium of the invention comprises a neurobasal medium, the SMAD inhibitors 4[4-(1,3-benzodioxol-5-yl)-5-(2-pyridinyl)-1H-imidazol-2-yl]benzamide (SB431542) and 4-(6-(4-(piperazin-1-yl)phenyl)pyrazolo[1,5-a]pyrimidin-3-yl)quinolone hydrochloride (LDN-193189) and the nitric oxide synthase inhibitor 1-(2-Trifluoromethylphenyl) lmidazole (TRIM) and B27 supplement.

Typically, the culture medium of the invention is free of serum and free of serum extract.

In a preferred embodiment, the culture medium of the invention is free of animal-derived substances. In a preferred embodiment, the culture medium of the invention consists essentially of synthetic compounds, compounds of human origin and water. Advantageously, said culture medium can be used for culturing cells according to good manufacturing practices (under “GMP” conditions).

Kits

The present invention provides a kit for the neuronal differentiation of a cell. In one embodiment, the kit provides at least two inhibitors of SMAD signaling and at least one inhibitor of NOS. In addition to inhibitors of SMAD signaling and NOS, the kits of the present invention may further comprise one or more of the following: a culture medium (such as the cell culture medium described herein), at least one cell culture medium supplement, an agent for inhibiting or increasing expression of one or more gene products, and at least one agent for detecting expression of a marker of neuronal differentiation.

In one embodiment, the at least two SMAD inhibitors selected from SB431541, SB431542, LDN193189, SB505124, LY2157299, DMH1, SIS3 and Noggin and at least one NOS inhibitor selected from SB431541, SB431542, LDN193189, SB505124, LY2157299, DMH1, SIS3 and Noggin and at least one NOS inhibitor selected from any one of Diphenyleneiodonium Chloride, Dexamethasone, 1-Pyrrolidinecarbodithioic Acid, 7-Nitroindazole, 1400W, 1-Amino-2-hydroxyguanidine, p-Toluenesulfonate, S-Methylisothiourea, 1,3-PBITU, L-NIL, TRIM, PPM-18, The Nitric Oxide Synthase, Neuronal Inhibitor I, Chlorpromazine, Spermidine, N^{G}-Nitro-L-arginine, Aminoguanidine, S-Methyl-L-thiocitrulline, S-Methylisothiourea, Zinc (II) Protoporphyrin IX, MEG, Bromocriptine Mesylate, Melatonin, L-Thiocitrulline, N^{G},N^{G}-Dimethyl-L-arginine, N^{G}-Propyl-L-arginine, SKF-525A, Haloperidol, N^{G}-Monomethyl-D-arginine, 2-Ethyl-2-thiopseudourea, L-N ⁵ -(1-Iminoethyl)ornithine, Caveolin-1 Scaffolding Domain Peptide and p-Nitroblue Tetrazolium Chloride.

In another embodiment, the kit comprises the SMAD inhibitors SB431542 and LDN-193189 and the nitric oxide synthase inhibitor TRIM.

In another embodiment, the kit further comprises a cell selected from the group consisting of a stem cell, progenitor cell, a dedifferentiated cell, neural stem cell, neural progenitor cell, or primary olfactory cell. In another embodiment, the cell is a primary olfactory cell is from an olfactory neurosphere.

Gene Expression Analysis

The methods and cells produced according to the methods of this invention are also of interest in identifying expression patterns of transcripts and newly synthesized proteins that are characteristic for neural precursor cells and neurons, and may assist in directing the differentiation pathway or facilitating interaction between cells. Expression patterns of the differentiated cells are obtained and compared with the cells from which they have been differentiated (e.g. neural stem or progenitor cells, ONS) or control cell lines.

Suitable methods for comparing expression at the protein level include the immunoassay or immunohistochemistry techniques described herein. Suitable methods for comparing expression at the level of transcription are well known to those of skill in the art and can include methods of differential display of mRNA (Liang, Peng, et al., Cancer Res. 52:6966, 1992), whole-scale sequencing of cDNA libraries, and matrix array expression systems.

Identifying expression products for use in characterizing and effecting neuronal differentiation of cells of this invention involves analyzing the expression level of RNA, protein, or other gene product in a first cell type, such as a neural stem or progenitor cell, or a cell capable of differentiating along the neuronal or glial pathway; then analyzing the expression level of the same product in a control cell type; comparing the relative expression level between the two cell types, (typically normalized by total protein or RNA in the sample, or in comparison with another gene product expected to be expressed at a similar level in both cell types, such as a house-keeping gene); and then identifying products of interest based on the comparative expression level.

Alternatively, the effects of a gene product of interest may be identified by subjecting a cell to the differentiation method of the present invention wherein the expression of a gene product of interest in the cell has been modified. For example the expression of the gene product of interest may be inhibited or impaired (e.g. the gene product is non-functional or has impaired or reduced function) or increased or overexpressed or expression of a gene product of interest having enhanced function.

In another embodiment, the effects of a gene product of interest on neuronal function may be identified by modifying the expression of the gene product in a cell produced according to the methods of the invention produced according the present invention. For example, a neuron produced according to the methods of the invention may be used as a model of neurodegenerative disease wherein the neuron is modified such that the expression of a gene product of interest is inhibited, impaired or modified such as by knock-down, knock in, over-expression or gene editing to reduce, increase or modify the expression of a gene product of interest.

A cell subjected to the differentiation process of the invention may be obtained from a subject who has inhibited, impaired or increased expression of a gene product of interest (such as through a gene mutation) which is associated with a neurodegenerative disorder or a disorder of the peripheral nervous system. Alternatively, a cell subjected to the differentiation process of the present invention may be derived from a healthy subject not characterized as having a neurodegenerative disorder or a disorder of the peripheral nervous system, wherein expression of a functional gene product of interest in the cell manipulated such that expression is impaired, inhibited or silenced, such as through the use of an inhibitory molecule (e.g. siRNA, miRNA etc.) or the introduction of a mutation, or the expression may be enhanced or increased. The effects of the expression of the gene product of interest on neuronal differentiation and function may then be identified via comparison to control cells.

The means for inhibiting, silencing (knock-down), introducing a mutation into (gene editing) or increasing (knock-in) or overexpressing a gene of interest will be known to the skilled addressee.

In one embodiment, the present invention provides a method for identifying an effect of a gene product on neuronal differentiation or neuronal function, comprising inhibiting SMAD signaling and NOS in a cell and measuring differentiation and/or function, wherein the cell is a neural stem or progenitor cell or ONS-derived cell obtained from a subject who has a neurodegenerative disorder or a disorder of the peripheral nervous system, wherein the neural stem or progenitor cell or primary olfactory cell displays one or more of the following: i) inhibited expression of a gene product of interest, ii) expression of a gene product of interest with impaired function, iii) overexpression of a gene product of interest or iv) expression of a gene product of interest with enhanced function.

In another embodiment, the present invention provides a method for identifying an effect of a gene product on neuronal differentiation, comprising inhibiting SMAD signaling and NOS in a cell and measuring differentiation, wherein the cell is a neural stem or progenitor cell or ONS-derived cell obtained from a healthy subject, and wherein the cell has been modified to display one or more of the following: i) inhibited expression of a gene product of interest, ii) expression of a gene product of interest with impaired function, iii) overexpression of a gene product of interest or iv) expression of a gene product of interest with enhanced function.

One skilled in the art appreciates that the effects of a candidate gene product on neuronal differentiation performed according to the methods disclosed herein is typically compared to a corresponding control cell (e.g. a cell which does not display i) inhibited expression of a gene product of interest, ii) expression of a gene product of interest with impaired function, iii) increased expression or overexpression of a gene product of interest or iv) expression of a gene product of interest with enhanced function).

Drug Screening

The methods and neurons produced according to the methods disclosed herein can be used to screen for agents (such as solvents, small molecule drugs, peptides, polynucleotides) or environmental conditions (such as culture conditions or manipulation) that affect neuronal differentiation, cell viability or function.

In some applications, a stem cell, progenitor cell, a dedifferentiated cell, neural stern cell, neural progenitor cell, or primary olfactory cell (e.g. derived from an ONS) is used to screen factors that promote maturation into neural cells, or promote proliferation and maintenance of such cells in long-term culture for later neuronal differentiation. For example, candidate maturation factors or growth factors are tested by adding them to one or more cells in different wells, and then determining any change in the expression of a gene product of interest or any phenotypic change that results, according to desirable criteria for further culture and/or use of the cells.

Other screening applications of this invention relate to the testing of candidate agents for their effect on neural function (e.g. therapeutic, neuroprotective, or neurotoxic agents). Screening may be done either because the agent is designed to have a therapeutic effect on neural cells, or because a compound designed to have effects elsewhere may have unintended side effects on the nervous system. For example, candidate agents are tested by adding them to one or more cells in different wells, and then determining any change in the expression of a gene product of interest and/or any phenotypic change that results, according to desirable criteria. The screening can be conducted using any neurons produced according to the methods disclosed herein.

In a preferred embodiment, screening of candidate agents is performed using one or more neurons produced according to the methods disclosed herein wherein the neurons are derived from a neural stem or progenitor cell or primary olfactory cell (e.g. derived from an ONS) obtained from a subject who has a neurodegenerative disorder or a disorder of the peripheral nervous system.

In another embodiment, screening of candidate agents is performed using one or more neurons produced according to the methods disclosed herein wherein the neurons are derived from a neural stem or progenitor cell or primary olfactory cell obtained from a healthy subject who does not have a neurodegenerative disorder or a disorder of the peripheral nervous system.

In another embodiment, screening of candidate agents is performed using one or more neurons produced according to the methods disclosed herein, wherein the neurons are derived from a neural stem or progenitor cell or primary olfactory cell obtained from a healthy subject or a subject who has a neurodegenerative disorder or a disorder of the peripheral nervous system, wherein the neural stem or progenitor cell or ONS derived cell is manipulated to display i) inhibited expression of a gene product of interest, ii) expression of a gene product of interest with impaired function, iii) increased expression or overexpression of a gene product of interest or iv) expression of a gene product of interest with enhanced function.

In another embodiment, screening of candidate agents is performed using one or more neurons produced according to the methods disclosed herein, wherein the neurons are derived from a neural stern or progenitor cell or primary olfactory cell obtained from a healthy subject or a subject who has a neurodegenerative disorder or a disorder of the peripheral nervous system, wherein the neuron is manipulated to display i) inhibited expression of a gene product of interest, ii) expression of a gene product of interest with impaired function, iii) increased expression or overexpression of a gene product of interest or iv) expression of a gene product of interest with enhanced function.

One skilled in the art appreciates that the effects of a candidate agent on a neuron produced according to the methods disclosed herein is typically compared to a corresponding control cell in the absence of the candidate agent.

Candidate agents which may also be used in the discovery and development of a therapeutic compound for the treatment of a neurodegenerative disorder include small molecules, peptides, peptide mimetics, polypeptides, and nucleic acid molecules. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques. Small molecules of the invention preferably have a molecular weight below 2,000 daltons, more preferably between 300 and 1,000 daltons, and most preferably between 400 and 700 daltons. It is preferred that these small molecules are organic molecules.

In general, candidate agents are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or agent is not critical to the screening procedure(s) of the invention. Agents used in screens may include known agents (for example, known therapeutics used for other diseases or disorders). Alternatively, virtually any number of unknown chemical extracts or agent can be screened using the methods described herein. Examples of such extracts or agents include, but are not limited to, plant-, fungal-, prokaryotic- or animal-based extracts, fermentation broths, and synthetic agents, as well as modification of existing agents.

Numerous methods are also available for generating random or directed synthesis (e.g., semi-synthesis or total synthesis) of any number of chemical agents, including, but not limited to, saccharide-, lipid-, peptide-, and nucleic acid-based agent. Synthetic compound libraries are commercially available from Brandon Associates (Merrimack, N.H.) and Aldrich Chemical (Milwaukee, Wis.). Alternatively, a chemical agent to be used as candidate agent can be synthesized from readily available starting materials using standard synthetic techniques and methodologies known to those of ordinary skill in the art. Synthetic chemistry transformations and protecting group methodologies (protection and deprotection) useful in synthesizing the agent identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Alternatively, libraries of natural agents in the form of bacterial, fungal, plant, and animal extracts are commercially available from a number of sources, including Biotics (Sussex, UK), Xenova (Slough, UK), Harbor Branch Oceanographic Institute (Ft. Pierce, Fla.), and PhannaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91 : 11422, 1994; Zuckennann et al., J. Med. Chem. 37:2678, 1994; Cho et al., Science 261 :1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37: 1233, 1994. Furthermore, if desired, any library or compound is readily modified using standard chemical, physical, or biochemical methods.

Libraries of agents may be presented in solution (e.g., Houghten, Biotechniques 13:412-421. 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc. Natl. Acad. Sci. USA 89: 1865-1869, 1992) or on phage (Scott and Smith, Science 249:386-390, 1990; Devlin, Science 249:404-406, 1990; Cwirla et al., Proc. Natl. Acad. Sci. 87:6378-6382, 1990; Felici, J. Mol. Biol. 222:301 -310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract of interest is identified, further fractionation of the positive lead extract is necessary to isolate chemical constituents responsible for the observed effect. Thus, the goal of the extraction, fractionation, and purification process is the careful characterization and identification of a chemical entity within the crude extract may be used for the prevention or treatment of a neurodegenerative disorder in a subject or regeneration or repair of the CNS or peripheral nervous system. Methods of fractionation and purification of such heterogeneous extracts are known in the art. If desired, agents shown to be useful as therapeutics for the prevention or treatment of a neurodegenerative disorder in a subject or regeneration or repair of the CNS or peripheral nervous system are chemically modified according to methods known in the art.

In another embodiment, a chemical entity discovered to have medicinal value using the methods described herein is useful as a drug or as information for structural modification of an existing agent, e.g., by rational drug design. For therapeutic uses, the compositions or agents identified using the methods disclosed herein may be administered systemically, for example, formulated in a pharmaceutically-acceptable carrier. Preferable routes of administration include, for example, topical, oral, subcutaneous, intravenous, intraperitoneally, intramuscular, or intradermal injections that provide continuous, sustained levels of the drug in the patient. Treatment of human patients or other animals will be carried out using a therapeutically effective amount of a candidate agent in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and the clinical symptoms. Generally, amounts will be in the range of those used for other agents used in the prevention or treatment of a neurodegenerative disorder in a subject or regeneration or repair of the CNS or peripheral nervous system in a subject, although in certain instances lower amounts may be needed because of the increased specificity of the compound. A compound is administered at a dosage that controls the clinical or physiological symptoms as determined by a diagnostic method known to one skilled in the art, or using any assay that measures the transcriptional activation of a gene associated with a neurodegenerative disorder in a subject or regeneration or repair of the CNS or peripheral nervous system in a subject.

The invention also includes novel agents identified by the above-described screening assays. Optionally, such agents are characterized in one or more appropriate animal models to determine the efficacy of the compound for the treatment of a neurodegenerative disorder. Desirably, characterization in an animal model can also be used to determine the toxicity, side effects, or mechanism of action of treatment with such a compound. Furthermore, a novel agent identified in any of the above-described screening assays may be used for the treatment of a neurodegenerative disorder in a subject. Such agents are useful alone or in combination with other conventional therapies known in the art.

Pharmaceutical Compositions and Therapeutic Methods

The present invention also provides a pharmaceutical composition comprising a neuron produced according to the methods disclosed herein or a population of neurons according to the invention or a population comprising a combination of a neuron of the invention and the precursor from which the neuron may be derived. The pharmaceutical composition may generally include one or more pharmaceutically acceptable and/or approved carriers, additives, antibiotics, preservatives, adjuvants, diluents and/or stabilizers. Such auxiliary substances can be water, saline, glycerol, ethanol, wetting or emulsifying agents, pH buffering substances, or the like. Suitable carriers are typically large, slowly metabolized molecules such as proteins, polysaccharides, polylactic acids, polyglycollic acids, polymeric amino acids, amino acid copolymers, lipid aggregates, or the like. This pharmaceutical composition can contain additional additives such as mannitol, dextran, sugar, glycine, lactose or polyvinylpyrrolidone or other additives such as antioxidants or inert gas, stabilizers or recombinant proteins (e. g. human serum albumin) suitable for in vivo administration.

As used herein, the term “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Another aspect of the invention relates to a population of neurons of the invention as described herein, for use in treating a neurodegenerative disease or an injury to the central or peripheral nervous system. The invention also relates to a method for treating a neurodegenerative disease or an injury to the central or peripheral nervous system comprising the step of administering a pharmaceutically effective amount a population of neurons as described above optionally together with of a population of neural precursors to a patient in need thereof.

In the context of the invention, the term “treating” or “treatment”, as used herein, refers to a method that is aimed at delaying or preventing the onset of a pathology, at reversing, alleviating, inhibiting, slowing down or stopping the progression, aggravation or deterioration of the symptoms of the pathology, at bringing about ameliorations of the symptoms of the pathology, and/or at curing the pathology.

As used herein, the term “pharmaceutically effective amount” refers to any amount of neural precursors or neurons according to the invention (or a population thereof or a pharmaceutical composition thereof) that is sufficient to achieve the intended purpose. Effective dosages and administration regimens can be readily determined by good medical practice based on the nature of the pathology of the subject, and will depend on a number of factors including, but not limited to, the extent of the symptoms of the pathology and extent of damage or degeneration of the tissue or organ of interest, and characteristics of the subject (e.g., age, body weight, gender, general health, and the like).

For therapy, neural precursors, neurons produced according to a method described herein and combinations thereof and pharmaceutical compositions according to the invention may be administered via any appropriate route. The dose and the number of administrations can be optimized by those skilled in the art in a known manner.

These and other aspects of the invention are illustrated by the following non-limiting examples. It should be appreciated that in some aspects one or more embodiments described in the examples may be generally applicable in combination with one or more embodiments described above.

### EXAMPLE 1

### Characterization of Olfactory Primary Cultures and the Effects of Growth Factors on Olfactory Primary Cultures

Isolation and Culture of Human Primary Olfactory Cells

Adult olfactory biopsies were obtained by routine nasal surgical methods and olfactory cells were obtained using methods well known to those of skill in the art, such as those previously described by Murrell, W., F. Feron, A. Wetzig, N. Cameron, K. Splatt, B. Bellette, J. Bianco, C. Perry, G. Lee and A. Mackay-Sim (2005). “Multipotent stem cells from adult olfactory mucosa.” Dev Dyn 233(2): 496-515.

Cells were maintained in standard culture media, which consists of DMEM (GIBCO) with 10% FBS (GIBCO) and pen/strep (10000 U/10000 μg per mL). Cells were maintained at 37° C. with 5% CO₂. Cultures were passaged at confluence. All experiments were conducted using cells between passages 6 and10.

Preparation of Surfaces for Differentiation

Glass coverslips were treated with 67% nitric acid over night, and baked at 220° C. for 8 h before being used for differentiation. Tissue culture prepared surfaces do not need to be treated this way. Treated coverslips were then coated with poly-D-lysine (75 μg/mL)(PDL) in 0.1M Borate buffer overnight at room temperature and wash 3 times for 2 h each before further coating the PDL surfaces with 1:100 laminin at 37° C. overnight.

Differentiation

For olfactory primary cell culture differentiation, the following 4 conditions were used:
Condition 1: Neural basal media (Invitrogen)+pen/strep+1×B27+100 μM TRIM;
Condition 2: Neural basal media+pen/strep+1×B27+10 μM SB431542 (SB)+100 nM LDN-193189 (LDN)+/−100 μM TRIM;
Condition 3: Neural basal media+pen/strep+1×B27+10 μM SB431542 (SB)+100 nM LDN-193189 (LDN)+5 pg/mL GDNF+20 pg/mL BDNF+200 uM L-ascorbic acid (AA)+/−100 μM TRIM;
Condition 4: Neural basal media+pen/strep+1×B27+20 pg/mL BDNF+200 uM L-ascorbic acid (AA)+100 μM TRIM. Cells were differentiated for 4-7 days before collected for analysis.

Immunostaining

Cells were fixed in 4% paraformaldehyde for 20 min and then blocked for 2 h at room temperature before incubated with the appropriate primary antibodies overnight at 4° C. The following primary antibodies were used: mouse β-III-tubulin (Sigma, 1:500), mouse N-cam (Dako, 1:100), mouse GFAP (Merk, 1:500), chicken Sox1 (Merk, 1:500), rabbit Nestin (Merk, 1:500), rabbit Neurofilament-Light (Merk, 1:500), rabbit Synapsin (Calbiochem, 1:500) and sheep Olfactory Marker Protein (Abcam, 1:500). Appropriate secondary antibodies were added and incubated for lh before samples were washed and mounted using Prolong Gold (Invitrogen) and analyzed using either a fluorescent microscope (Nikon) or a confocal microscope (Leica). All secondary antibodies were from Molecular Probes and diluted at 1:1000, except for anti-sheep secondary antibody, which was from Dako and diluted at 1:500.

Results

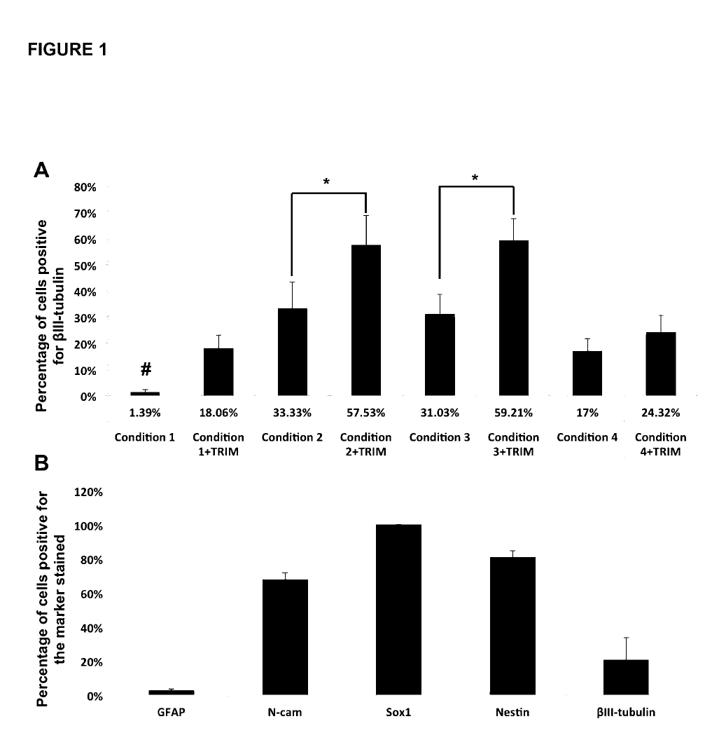

Olfactory primary cell cultures were characterized by immunostaining of some of the common neuronal markers. Almost all cells expressed early neuronal progenitor markers Sox 1 and Nestin, with around 20% of the cells expressed β-III-tubulin. Only less than 5% of the cells expressed the astrocyte marker GFAP. The majority of the olfactory primary cells exhibited an early neuronal phenotype (FIG. 1A).

The culture of olfactory primary cells on PDL and laminin coated surfaces in serum-free conditions did not yield cells with a typical neuronal morphology. However, it was observed that the addition of the two Smad4 inhibitors SB and LDN resulted in the increased number of β-III-tubulin positive cells in culture compared to samples cultured without the inhibitors. Moreover, the addition of the nitric oxide synthase inhibitor TRIM resulted in a further increase in β-III-tubulin positive cells in all conditions (FIG. 1B). Data in FIG. 1 are presented as mean±SD *: P<0.05, #: P<0.05 compared to all other samples, n=3 independent experiments.

### EXAMPLE 2

### Olfactory Neurospheres Provide a More Suitable Starting Material Compared to Olfactory Primary Cultures and Expanded Spheres for Neuronal Differentiation

Primary olfactory cells have the ability to form olfactory neurospheres (ONS) under serum-free conditions with the supplementation of EGF and FGF2.

Neurosphere Formation

Primary olfactory cells were dissociated into single cells and seeded onto Poly L-Lysine (Sigma) coated tissue culture dishes at 20,000 cells/cm² in sphering media, which consists of DMEM+1×ITS+50 μg/mL EGF+25 μg/mL FGF2. Cells were then incubated at 37° C. with 5% CO₂ until neurospheres started to form. This normally takes 2-3 days. Small neurospheres (<200 μm in diameter) were manually picked under a dissecting microscope (Leica) and collected for differentiation.

Preparation of Surfaces for Differentiation

Glass coverslips were treated with 67% nitric acid overnight, and baked at 220° C. for 8 h before used for differentiation. Tissue culture prepared surfaces do not need to be treated this way. Treated coverslips were then coated with poly-D-lysine (75 μg/mL)(PDL) in 0.1M Borate buffer overnight at room temperature and wash 3 times for 2 h each before further coating the PDL surfaces with 1:100 laminin at 37° C. overnight.

Differentiation

For olfactory neurosphere differentiation experiments, cells were cultured in ONS differentiation media, which consists of neural basal media+1×B27+1×L-glutamine+pen/strep+1×β-mercaptoethanol+AA+BDNF supplemented with combinations of SB, LDN and TRIM. Cells were differentiated for 3-4 days before collected for analysis. Media was changed on the second day.

RT-qPCR

RT-qPCR was performed on cDNA samples with a Rotor-Gene 6000 (Corbett) using SYBER green based assays (Qiagen). All samples were normalized to GAPDH and the relative expression of each gene was determined using the delta-delta CT method. The primer sequences used are as follows:

GAPDH,
F:
(SEQ ID NO. 1)
5′-ACAGTCAGCCGCATCTTCTT-3′,
R:
(SEQ ID NO. 2)
5′-ACGACCAAATCCGTTGACTC-3′;
N-Cam,
F:
(SEQ ID NO. 3)
5′-ATGGAAACTCTATTAAAGTGAACCTG-3′,
R:
(SEQ ID NO. 4)
5′-TAGACCTCATACTCAGCATTCCAGT-3′;
β-III-tubulin
F:
(SEQ ID NO. 5)
5′-CAAGTTCTGGGAAGTCATCAGTGA-3′,
R:
(SEQ ID NO. 6)
5′-CCGAGTCGCCCACGTAGTT-3′;
Nestin,
F:
(SEQ ID NO. 7)
5′-GCGTTGGAACAGAGGTTGGA-3′,
R:
(SEQ ID NO. 8)
5′-TGGGAGCAAAGATCCAAGAC-3′.

Results

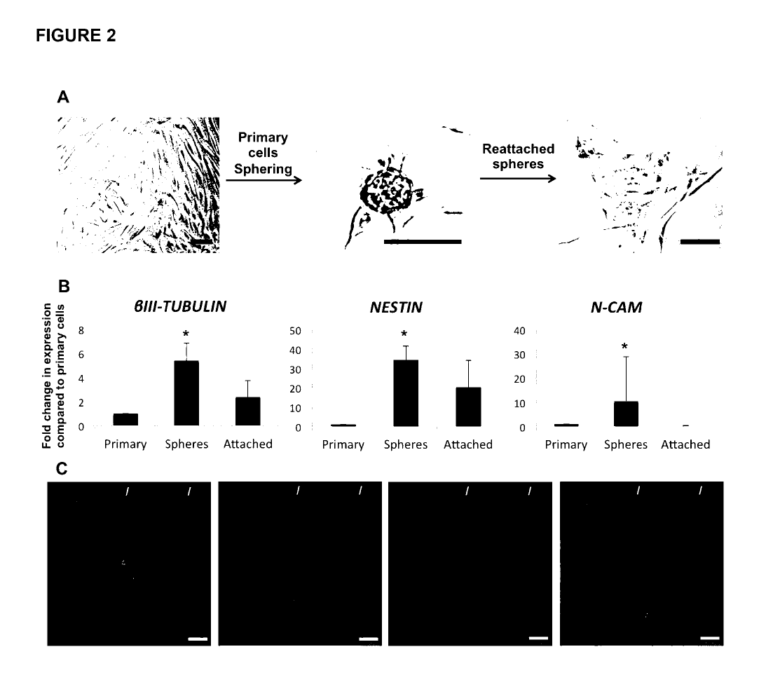

The ONS can then be re-attached to tissue culture dishes and cultured in the presence of serum as expanded spheres (FIG. 2A) (scale bar=100 μm). The mRNA levels of β-III-tubulin, Nestin and N-cam in the primary cells, ONS and expanded spheres were analyzed using RT-qPCR (FIG. 2B)(n=4 independent experiments. Data presented as mean±SD, Y-axis represents expression fold change compared to primary cells *: p<0.05. Elevated levels of β-III-tubulin, Nestin and N-Cam were observed in ONS compared to both primary cells and expanded spheres. Confocal microscopy analysis of ONS labelled with β-III-tubulin (green) and Synapsin (red) showed that almost all cells in the ONS expressed β-III-tubulin with a small percentage of the cells positive for Synapsin (FIG. 2C)(scale bar=25 μm).

### EXAMPLE 3

### Almost all Olfactory Neurospheres Exhibit Neuronal Phenotype after the Combined Treatment of SB, LDN and TRIM

The ability for ONS to respond to the treatments of SB, LDN and TRIM was tested. QPCR was performed according to the methods described in the foregoing examples. The data revealed showed that the combination of SB and LDN significantly reduced the mRNA level of SMAD4 compared to the cells cultured without SB or LDN. Compared to cells cultured in the absence of inhibitors, cells cultured with SB431542 alone showed no change in SMAD4 expression. The level of inhibition of SMAD4 with LDN193189 alone was 80%, whereas supplementation with SB and LDN yielded a level of inhibition of 92%.

Cells were also stained for Synapsin and OMP, a marker for mature olfactory neurons according to the methods described in the foregoing examples.

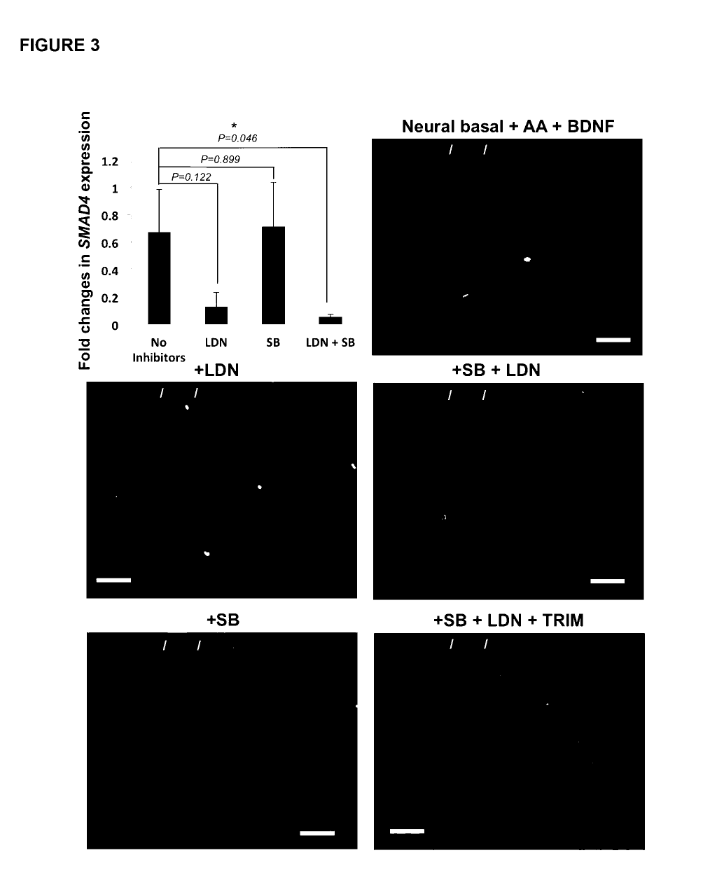

After 3-4 days of differentiation, cells from all treatment groups expressed Synapsin, however, the expression of OMP was only observed in a population of cells treated with a combination of SB, LDN and TRIM. Cells cultured in ONS differentiation media, which contains neural basal media supplemented with ascorbic acid and BDNF did not exhibit a typical neuronal morphology. With the addition of SB or LDN in the differentiation media, short neurites were observed in the majority of the cells in culture. Supplementation of the combination of SB and LDN resulted in cells with longer neurites and branching of the neurites can be observed in some cells. The supplementation of SB, LDN and TRIM resulted in cells with long neurite extensions compared to those treated with SB and LDN only (FIG. 3) (n=3 independent experiments. Scale bar=100 μm).

The supplementation of SB, LDN and TRIM also resulted in highly efficient neuronal differentiation wherein 83.02%+13.13% of ONS to differentiated into neurons.

### EXAMPLE 4

### Further Characterization of Olfactory Neurosphere Derived Neurons

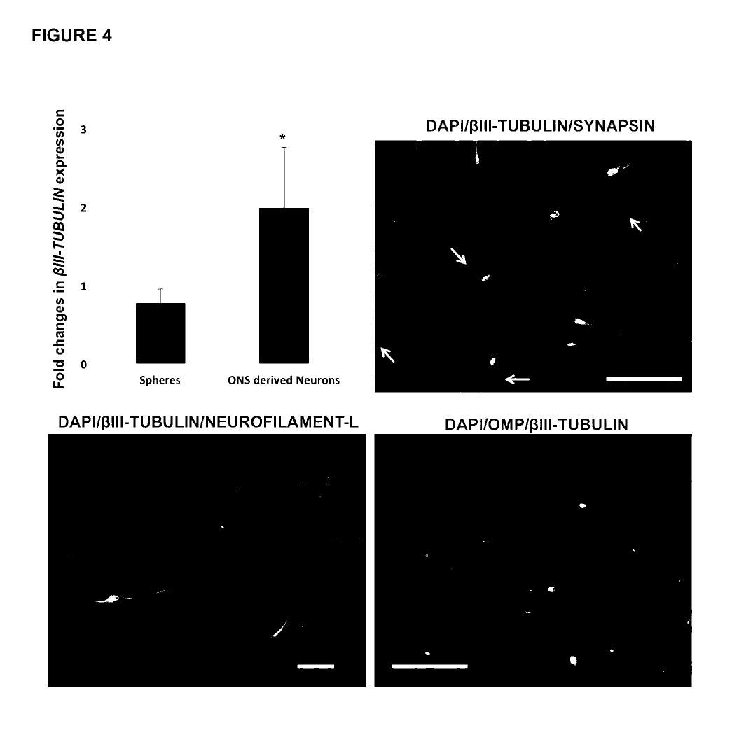

The ONS derived neurons were further characterized using RT-qPCR and Immunostaining techniques as described in the foregoing examples. RT-qPCR analysis showed that the neurons expressed higher levels of β-III-tubulin compared to ONS (FIG. 4)(n=4 independent experiments). Data presented as mean± standard deviation. Immunostaining showed the expression of βIII-Tubulin, Synapsin, Neurofilament-L and OMP. Arrows indicate synaptic endings as judged by strong SYNAPSIN staining at the end of the neurites (scale bar=100 μm).

### REFERENCES

Abrahamsen, G., Y. Fan, N. Matigian, G. Wali, B. Bellette, R. Sutharsan, J. Raju, S. A. Wood, D. Veivers, C. M. Sue and A. Mackay-Sim (2013). “A patient-derived stem cell model of hereditary spastic paraplegia with SPAST mutation.” Dis Model Mech 6(2): 489-502.
Chambers, S. M., C. A. Fasano, E. P. Papapetrou, M. Tomishima, M. Sadelain and L. Studer (2009). “Highly efficient neural conversion of human ES and iPS cells by dual inhibition of SMAD signaling.” Nat Biotechnol 27(3): 275-280.
lmai, Y., M. Soda, H. Inoue, N. Hattori, Y. Mizuno and R. Takahashi (2001). “An unfolded putative transmembrane polypeptide, which can lead to endoplasmic reticulum stress, is a substrate of Parkin.” Cell 105(7): 891-902.
Murrell, W., F. Feron, A. Wetzig, N. Cameron, K. Splatt, B. Bellette, J. Bianco, C. Perry, G. Lee and A. Mackay-Sim (2005). “Multipotent stem cells from adult olfactory mucosa.” Dev Dyn 233(2): 496-515.
Murrell, W., A. Wetzig, M. Donnellan, F. Féron, T. Burne, A Meedeniya, J. Kesby, J. Bianco, C. Perry, P. Silburn and A. Mackay-Sim (2008). “Olfactory mucosa is a potential source for autologous stem cell therapy for Parkinson's disease.” Stem Cells 26(6): 2183-2192.
Park, T. I., H. Monzo, E. W. Mee, P. S. Bergin, H. H. Teoh, J. M. Montgomery, R. L. Faull, M. A. Curtis and M. Dragunow (2012). “Adult human brain neural progenitor cells (NPCs) and fibroblast-like cells have similar properties in vitro but only NPCs differentiate into neurons.” PLoS One 7(6): e37742. doi: 37710.31371/journal.pone.0037742.
Stewart, R., S. Kozlov, N. Matigian, G. Wali, M. Gatei, R. Sutharsan, B. Bellette, A. Wraith-Kijas, J. Cochrane, M. Coulthard, C. Perry, K. Sinclair, A. Mackay-Sim and M. F. Lavin (2013). “A patient-derived olfactory stern cell disease model for ataxia-telangiectasia.” Hum Mol Genet 22(12): 2495-2509.
Sülz, L., G. Astorga, B. Bellette, R. Iturriaga, A. Mackay-Sim and J. Bacigalupo (2009). “Nitric oxide regulates neurogenesis in adult olfactory epithelium in vitro.” Nitric Oxide 20(4): 238-252.
Valente, E. M., P. M. Abou-Sleiman, V. Caputo, M. M. Muqit, K. Harvey, S. Gispert, Z. Ali, D. Del Turco, A. R. Bentivoglio, D. G. Healy, A. Albanese, R. Nussbaum, R. Gonzalez-Maldonado, T. Deller, S. Saivi, P. Cortelli, W. P. Gilks, D. S. Latchman, R. J. Harvey, B. Dallapiccola, G. Auburger and N. W. Wood (2004). “Hereditary early-onset Parkinson's disease caused by mutations in PINK1.” Science 304(5674): 1158-1160. Epub 2004 Apr 1115.
Wetzig, A. A. Mackay-Sim and W. Murrell (2011). “Characterization of olfactory stem cells. ” Cell Transplant 20(11-12): 1673-1691.

1. A method for producing a neuron comprising inducing neuronal differentiation of a cell, wherein neuronal differentiation in said cell is induced by inhibition of Small Mothers Against Decapentaplegic (SMAD) signaling and nitric oxide synthase (NOS) in said cell.

2. The method according to claim 1, wherein said inhibition of SMAD signaling occurs by contacting the cell with at least two SMAD inhibitors and inhibition of NOS occurs by contacting the cell with at least one NOS inhibitor.

3. The method according to claim 2, wherein the SMAD inhibitors are selected from any one of SB431541, 4-[4-(1,3-benzodioxol-5-yl)-5-(2-pyridinyl)-1H-imidazol-2-yl]benzamide (SB431542), 4-(6-(4-(piperazin-1-yl)phenyl)pyrazolo[1,5-a]pyrimidin-3-yl)quinolone hydrochloride (LDN193189), 2-(4-(benzo[d][1,3]dioxol-5-yl)-2-tert-butyl-1H-imidazol-5-yl)-6-methylpyridine (SB505124), 4-[2-(6-Methyl-pyridin-2-yl)-5,6-dihydro-4H-pyrrolo[1,2-b]pyrazol-3-yl]-quinoline-6-carboxylic acid amide (LY2157299), 4-[6-(4-Isopropoxyphenyl)pyrazolo[1,5-a]pyrimidin-3-yl]quinoline, 4-[6-[4-(1-Methylethoxy)phenyl]pyrazolo[1,5-a]pyrimidin-3-yl]-quinoline (DMH1), (2E)-1-(6,7-Dimethoxy-3,4-dihydro-1H-isoquinolin-2-yl)-3-(1-methyl-2-phenyl-1H-pyrrolo[2,3-b]pyridin-3-yl)-propenone hydrochloride (SIS3) and Noggin.

4. The method according to claim 2, wherein the NOS inhibitor is selected from any one of Diphenyleneiodonium Chloride, Dexamethasone, 1-Pyrrolidinecarbodithioic Acid, 7-Nitroindazole, 1400W, 1-Amino-2-hydroxyguanidine, p-Toluenesulfonate, S-Methylisothiourea, S,S′-1,3-Phenylene-bis(1,2-ethanediyl)-bis-isothiourea.2HBr (1,3-PBITU), N6-(1-iminoethyl)-L-lysine (L-NIL), 1-(2-Trifluoromethylphenyl) Imidazole (TRIM), N-(1,4-dihydro-1,4-dioxo-2-naphthalenyl)-benzamide (PPM-18), The Nitric Oxide Synthase Neuronal Inhibitor I, Chlorpromazine, Spermidine, N^{G}-Nitro-L-arginine, Aminoguanidine, S-Methyl-L-thiocitrulline, S-Methylisothiourea, Zinc (II) Protoporphyrin IX, Mercaptoethylguanidine (MEG), Bromocriptine Mesylate, Melatonin, L-Thiocitrulline, N^{G},N^{G}-Dimethyl-L-arginine, N^{G}-Propyl-L-arginine, α-phenyl-α-propyl-2-(diethylamino)ethyl ester-benzeneacetic (SKF-525A, Proadifen), Haloperidol, N^{G}-Monomethyl-D-arginine, 2-Ethyl-2-thiopseudourea, L-N⁵-(1-Iminoethyl)ornithine, Caveolin-1 Scaffolding Domain Peptide and p-Nitroblue Tetrazolium Chloride.

5. The method according to claim 2, wherein the SMAD inhibitors are SB431542 and LDN193189 and the nitric oxide synthase inhibitor is TRIM.

6. The method according to claim 5 wherein the cell is contacted with a medium comprising SB431542 at a concentration of about 5 μM to about 100 μM, LDN193189 at a concentration of about 5 nM to about 500 nM, and TRIM at a concentration of about 10 μM to about 1000 μM.

7. The method according to claim 6 wherein the concentration of SB431542 is about 10 μM, the concentration of LDN193189 is about 100 nM, and the concentration of TRIM is about 100 μM.

8. The method according to claim 1, wherein said cell is isolated from a human.

9. The method according to claim 8, wherein said cell is selected from the group consisting of a stem cell, progenitor cell, a dedifferentiated cell, neural stem cell, neural progenitor cell, or primary olfactory cell.

10. The method according to claim 9, wherein the primary olfactory cell is from an olfactory neurosphere.

11. The method for producing a neuron according to claim 1 comprising the steps of:
i. culturing one or more primary olfactory cells from a subject in culture conditions to form an olfactory neurosphere;
ii. isolating said neurosphere; and
iii. inducing neuronal differentiation in one or more cells of said neurosphere by culturing said one or more cells under conditions which inhibit SMAD signaling and NOS;
wherein neuronal differentiation is achieved after about 3 to 4 days following step (iii).

12-18. (canceled)

19. A neuron produced according to the method of claim 1.

20-22. (canceled)

23. A kit for inducing neuronal differentiation of a cell, comprising at least two SMAD inhibitors and at least one NOS inhibitor.

24. The kit according to claim 23, wherein the SMAD inhibitors are selected from any one of SB431541, 4-[4-(1,3-benzodioxol-5-yl)-5-(2-pyridinyl)-1H-imidazol-2-yl]benzamide (SB431542), 4-(6-(4-(piperazin-1-yl)phenyl)pyrazolo[1,5-a]pyrimidin-3-yl)quinolone hydrochloride (LDN193189), 2-(4-(benzo[d][1,3]dioxol-5-yl)-2-tert-butyl-1H-imidazol-5-yl)-6-methylpyridine (SB505124), 4-[2-(6-Methyl-pyridin-2-yl)-5,6-dihydro-4H-pyrrolo[1,2-b]pyrazol-3-yl]-quinoline-6-carboxylic acid amide (LY2157299), 4-[6-(4-Isopropoxyphenyl)pyrazolo[1,5-a]pyrimidin-3-yl]quinoline, 4-[6-[4-(1-Methylethoxy)phenyl]pyrazolo[1,5-a]pyrimidin-3-yl]-quinoline (DMH1), (2E)-1-(6,7-Dimethoxy-3,4-dihydro-1H-isoquinolin-2-yl)-3-(1-methyl-2-phenyl-1H-pyrrolo[2,3-b]pyridin-3-yl)-propenone hydrochloride (SIS3) and Noggin.

25. The kit according to claim 23, wherein the NOS inhibitor is selected from any one of Diphenyleneiodonium Chloride, Dexamethasone, 1-Pyrrolidinecarbodithioic Acid, 7-Nitroindazole, 1400W, 1-Amino-2-hydroxyguanidine, p-Toluenesulfonate, S-Methylisothiourea, S,S′-1,3-Phenylene-bis(1,2-ethanediyl)-bis-isothiourea.2HBr (1,3-PBITU), N6-(1-iminoethyl)-L-lysine (L-NIL), 1-(2-Trifluoromethylphenyl) Imidazole (TRIM), N-(1,4-dihydro-1,4-dioxo-2-naphthalenyl)-benzamide (PPM-18), The Nitric Oxide Synthase Neuronal Inhibitor I, Chlorpromazine, Spermidine, N^{G}-Nitro-L-arginine, Aminoguanidine, S-Methyl-L-thiocitrulline, S-Methylisothiourea, Zinc (II) Protoporphyrin IX, Mercaptoethylguanidine (MEG), Bromocriptine Mesylate, Melatonin, L-Thiocitrulline, N^{G},N^{G}-Dimethyl-L-arginine, N^{G}-Propyl-L-arginine, α-phenyl-α-propyl-2-(diethylamino)ethyl ester-benzeneacetic (SKF-525A, Proadifen), Haloperidol, N^{G}-Monomethyl-D-arginine, 2-Ethyl-2-thiopseudourea, L-N⁵-(1-Iminoethyl)ornithine, Caveolin-1 Scaffolding Domain Peptide and p-Nitroblue Tetrazolium Chloride.

26. The kit according to claim 23, wherein the SMAD inhibitors are SB431542 and LDN193189 and the nitric oxide synthase inhibitor is TRIM.

27. The kit according to claim 23, further comprising a cell selected from the group consisting of a stem cell, progenitor cell, a dedifferentiated cell, neural stem cell, neural progenitor cell, or primary olfactory cell.

28. (canceled)

29. The kit according to claim 23, further comprising agent for detecting expression of one or more markers of neuronal differentiation.

30. The kit according to claim 23, further comprising a cell culture medium.

31. The kit according to claim 30, further comprising one or more of B27, L-glutamine, β-mercaptoethanol, Amino acids and BDNF. 32-43. (Cancelled)


SEQUENCE LISTING (8 sequences)

SEQ ID NO: 1 - DNA, 20 bp, Artificial Sequence
  Description of Artificial Sequence Synthetic primer
acagtcagccgcatcttctt

SEQ ID NO: 2 - DNA, 20 bp, Artificial Sequence
  Description of Artificial Sequence Synthetic primer
acgaccaaatccgttgactc

SEQ ID NO: 3 - DNA, 26 bp, Artificial Sequence
  Description of Artificial Sequence Synthetic primer
atggaaactctattaaagtgaacctg

SEQ ID NO: 4 - DNA, 25 bp, Artificial Sequence
  Description of Artificial Sequence Synthetic primer
tagacctcatactcagcattccagt

SEQ ID NO: 5 - DNA, 24 bp, Artificial Sequence
  Description of Artificial Sequence Synthetic primer
caagttctgggaagtcatcagtga

SEQ ID NO: 6 - DNA, 19 bp, Artificial Sequence
  Description of Artificial Sequence Synthetic primer
ccgagtcgcccacgtagtt

SEQ ID NO: 7 - DNA, 20 bp, Artificial Sequence
  Description of Artificial Sequence Synthetic primer
gcgttggaacagaggttgga

SEQ ID NO: 8 - DNA, 20 bp, Artificial Sequence
  Description of Artificial Sequence Synthetic primer
tgggagcaaagatccaagac